(19) United States
(12) Patent Application Publication
(10) Pub. No.: US 2024/0203531 A1
(43) Pub. Date: Jun. 20, 2024

(54) CELL TYPE ANNOTATION

(71) Applicant: FLUENT BIOSCIENCES INC., Watertown, MA (US)

(72) Inventors: Yigal Agam, Watertown, MA (US); Corey Hayford, Watertown, MA (US)

(21) Appl. No.: 18/544,824

(22) Filed: Dec. 19, 2023

Related U.S. Application Data
(60) Provisional application No. 63/433,795, filed on Dec. 20, 2022.

(51) Int. Cl. G16B 40/30; C12Q 1/6806; C12Q 1/6869

(52) U.S. Cl. CPC G16B 40/30; C12Q 1/6806; C12Q 1/6869

(57) ABSTRACT

The invention provides methods for annotating cell types using non-parametric statistical scoring of gene expression levels from RNA sequencing (RNA-seq). Expression levels for cells are measured by RNA-seq and a non-parametric statistic such as a Mann-Whitney U score or Wilcoxon score is generated for the expression levels and correlated to such scores from reference data from known cell types. When test cells in the RNA-seq data have a score that correlate highly with such a score from cells of a known type in the reference, those test cells are annotated as being of the known type from the reference.

### TECHNICAL FIELD

The disclosure relates to expression analysis with methods such as single-cell RNA-seq.

### BACKGROUND

Single-cell RNA-sequencing (scRNA-seq) refers to a variety of protocols that involve sequencing RNA from a cell and seeking to provide a measure of gene expression levels from the sequence data. Some approaches to scRNA-Seq rely on isolating cells into droplets with the potential to assay a large number of cells per experiment. Some droplet-based protocols include Drop-seq (described in Macosko, 2015, Highly parallel genome-wide expression profiling of individual cells using nanoliter droplets, Cell 161(5):1202-14, incorporated by reference) and inDrop (see Klein, 2015, Droplet barcoding for single-cell transcriptomics applied to embryonic stem cells, Cell 161(5):1187-201, incorporated by reference).

A number of factors make it difficult to provide the measure of gene expression levels from the sequence data. For example, the barcodes are subject to errors that occur during sequencing and amplification. Additionally, much of the sequence data comes from the amplification of stray nucleic acids in droplets that did not contain a cell or that contained a damaged cell or other debris. Additionally, it is difficult to identify, from RNA-seq data, all the cell types that were present in the sample.

### SUMMARY

The invention provides methods for annotating cell types using non-parametric statistical scoring of gene expression levels from RNA sequencing (RNA-seq). Expression levels for cells are measured by RNA-seq and a non-parametric statistic such as a Mann-Whitney U score or Wilcoxon score is generated for the expression levels and correlated to such scores from reference data from known cell types. When test cells in the RNA-seq data have a score that correlate highly with such a score from cells of a known type in the reference, those test cells are annotated as being of the known type from the reference. Methods of the invention work well with high-throughput single-cell RNA-seq (scRNA-seq) technologies, such as those scRNA-seq techniques that encapsulate cells into aqueous partitions and capture transcripts in the partitions using barcoded capture oligos.

Because high-throughput scRNA-seq can generate expression profiles for a large number of cells of various different cell types, methods of the invention provide for identifying the different cell types by clustering the cells based on expression levels and then annotating the cell clusters by cell type. Expression profiles for each cell in an experiment may be pre-processed (e.g., normalized, log transformed, gene selected, and/or scaled) and subject to principal component analysis (PCA). A suitable clustering operations, such creating a nearest neighbor graph in a PCA matrix and performing a clustering algorithm such as the Leiden algorithm on the nearest neighbor graph assigns the test cells into clusters of cells with similar expression profiles. Those clusters are compared to clusters in a reference dataset made in a similar manner from cells of known type. An insight of the present invention is that very good results are obtained by scoring differential gene expression in the clusters with a non-parametric statistic and correlating those scores from the test clusters to such scores calculated for the reference clusters.

Some prior art methods require that differential gene expression be fit to a parameterized probability distribution, in which a distribution is specified and/or one or more parameters of the distribution such as mean or variance are specified. An insight of the invention is that requiring a parameterized probability distribution for differential gene expression levels imposes artificial or unnecessarily limits on the data such as low dynamic range or poor signal-to-noise ratio. Here, gene expression data and, in particular, differential gene expression levels, are scored using a non-parametric statistical score such as the Mann-Whitney U score (or Wilcoxon rank sum), an Anderson-Darling test, a t-test, or a bootstrap method. Using a non-parametric statistical score allows for a much greater dynamic range in differential gene expression levels, compared to fitting to a parameterized probability distribution. When differential gene expression is scored for clusters in the test data and the reference (e.g., the U score), and that score is used to correlate test clusters to annotated reference clusters-annotated by their known cell type-the correlation is found to give good results in annotating the test clusters by cell type.

Thus, methods of the invention are used to annotate cells from scRNA-seq experiments by cell type. Using non-parametric statistical scores when making comparisons to an annotated reference increases the dynamic range of expression levels used in the annotation and improves signal-to-noise ratio. Those benefits improve the accuracy of the cell-type annotations and extend the applicability to a great variety of sample types, including clinical samples of a variety of tissue types and disease states as well as samples from other sources such as environmental, forensics, and pathogen detection. In fact, cell type annotation according to methods of the invention are conducive to automation and well suited to very high-throughput scRNA-seq experiments. In particular, methods of the invention are particularly well suited to scRNA-seq technologies that involve massive parallelism and throughput such as partition-based scRNA-seq platforms that perform simultaneous capture of large numbers of cells into aqueous partitions with target capture and some steps of sample preparation performed in the partitions. One example of a partition-based scRNA-seq platform that uses simultaneous capture of large numbers of cells into aqueous partitions involves combining template particles (such as hydrogel beads decorated with capture oligos) with cells in an aqueous fluid and shearing the fluid in the presence of an oil to simultaneously generate a large number of aqueous droplets in the oil, in which each droplet contains one of the template particles.

When shearing, or vortexing, a mixture including the particles, aqueous liquid, and oil, those particles operate like templates, each seeding the formation of one aqueous partition. By including cells from the sample at an appropriate concentrations, on average, all of the partitions will include zero or one cell. The energy from shearing or vortexing causes the partitions to all form essentially simultaneously and instantly. Those partitions may be referred to as particle-templated instant partitions, or PIPs. The creation of PIPs is a good way to encapsulate a very large number (e.g., >hundreds of thousands) of single cells into aqueous partitions that each contain one bead decorated with capture oligos. Those capture oligos can have sequences that hybridize to, and capture, RNA molecules (e.g., poly-T sequence, gene-specific primers, random hexamers, others, or combinations thereof). The capture oligos can also include barcodes including bead-specific barcodes (which operate as cell-identifying barcodes) and unique molecule identifiers (which as long as they are nearly unique provide a tool for deduplicating sequence reads and counting unique molecules from a cell). With those capture oligos, PIPs are well-suited to sample capture and library preparation for scRNA-seq. Due to the robustness and accuracy of cell-annotation methods of the invention, the cell annotation methods are particularly well-suited to the diverse cell and sample types and the high throughput and massive parallelism of scRNA-seq methods using cells in partitions such as PIPs. Thus the invention provides methods for annotating cells with their cell types from scRNA-seq data. Those methods are particularly well suited to scRNA-seq techniques that use aqueous partitions such as PIPs.

In certain aspects, the invention provides methods of identifying cell types. The methods include obtaining gene expression data for a plurality of cells; assigning the cells to clusters based on the gene expression data; determining non-parametric statistical scores of differential gene expression for each cluster versus other clusters; calculating correlations between the calculated score and similar scores for annotated clusters in a reference; and annotating each cluster with a cell type based on correlations to the annotated clusters. In some embodiments, each cluster is given an annotation in one of three categories such as (i) definitive, (ii) primary and secondary, and (iii) no assignment.

Determining the non-parametric statistical score may include performing a Mann-Whitney test to calculate a U score for genes of each cluster. The annotated clusters in the reference may also have reference U scores. The grouping may be done by creating a k-nearest neighbor graph in a PCA matrix of gene expression levels for the cells and then performing Leiden clustering on the graph. Preferably, the assigning, determining, calculating, and annotating are performed by a computer system comprising at least one processor coupled to a memory subsystem containing instructions executable by the processor to cause the computer system to perform the recited operations.

In some embodiments, the reference includes the annotated clusters of reference cells of known type, in which the annotated clusters were created by identifying reference genes with the highest differential expression among the reference cells and clustering the reference cells into the annotated clusters based on those reference genes. The reference may include reference U scores calculated by a non-parametrical statistical test (e.g., the Wilcoxon ranked sum) for each annotated cluster.

Methods may include performing the obtaining step by single-cell RNA-sequencing (scRNA-seq) to obtain scRNA-seq data and performing the determining step to produce the non-parametric statistical scores of differential gene expression for each cluster from the scRNA-seq data. In some embodiments, the tested cells are peripheral blood monocytes (PBMCs) and the clusters are annotated as B cells, CD4 memory T cells, CD4 naïve T cells, etc. Some embodiments of the methods use PIPs, and may include combining template particles (e.g., hydrogel particles linked to copies of a capture probe or oligo) with cells in a first fluid; shearing the first fluid (e.g., an aqueous media) in the presence of a second immiscible fluid (e.g., oil) to thereby generate a plurality of droplets substantially simultaneously, such that at least one droplet contains a single template particle and a single target cell; lysing the cell in the droplet to release mRNA molecules; and capturing the mRNA molecule onto the single template particle. Methods may include performing reverse transcription to yield a cDNA using a reverse transcriptase (RT), e.g., after breaking the droplets and releasing the particles into a bulk phase. In certain template-switching oligo (TSO) embodiments, the RT adds additional bases to a 3′ end the cDNA. In some embodiments, the reverse transcription leaves the cDNA linked to the particle, with the cDNA including first and second universal primer binding sites, at least one cell barcode, and at least one unique molecular identifier (UMI). Preferably the capture probe or oligo includes and provides one or more of a template switching segment, a UMI, a droplet barcode, and a universal primer nucleotide sequence. The template particles may have compartments, recesses, or pores that contain reagents such as one or more of a cell lysis reagent, a nucleic acid synthesis reagent, ions, co-factors, and a capture tag or oligonucleotide.

Methods may include quantifying the plurality of distinct mRNA molecules and optionally generating an expression profile for the cells after quantifying the plurality of distinct mRNA molecules. It may be preferable to preprocess expression data for the cells using one or a combination of (i) normalization; (ii) log transformation; (iii) gene selection; and (iv) scaling. Methods may be applied to (i) normalize expression levels within and for each cell (improving inter-cell comparisons). The methods may continue on to (ii) transform the resultant matrix by a function such as ln(x+1) to approximate a normal distribution. At a stage such as this, it may be preferable to (iii) select, from all of the genes, a subset of the genes with high variance (to greatly improve clustering). E.g., for every gene, the variance in expression across cells is calculated; the genes are then ranked by variance, and the top genes at the Nth percentile are selected. At this stage, it may be preferable to (iv) scale the feature columns (genes), e.g., so that each column has a mean of 0 and a standard deviation of 1. The optionally pre-processed gene expression data may be subject to principal component analysis (PCA), to reduce the dimensionality of data to a limited (e.g., 2, 3, 4 . . . ) number of principal components that best account for variance in the data. Performing PCA provides a PCA matrix, a matrix in which cells are positioned by gene expression level such that similar cells are more proximal to each other than to dissimilar cells. The PCA matrix may be subject to an automated process for defining clusters of similar cells.

Any suitable process may be used to identify clusters of similar cells from the PCT matrix (similar here refers to the real-world similarity of cells, meaning that cells will be grouped based cell type, e.g., all B cells with be together in a group that does not include natural killer cells). Suitable processes for identifying or defining cell clusters may include a community detection algorithm for example, or a method from the literature such as RaceID, SNN-Cliq, SINCERA, SEURAT, or SC3. Preferred embodiments involve constructing a k-nearest-neighbor (knn) graph from the PCA matrix and passing the graph to a Leiden algorithm to optimize cluster membership. The clustering process assigns the cells to clusters based on similarities among expression profiles. Those expression profiles across a cluster are subject to a non-parametric statistical scoring, such as calculation of the Mann-Whitney U score, and correlations are calculated between the U scores for each of the clusters and U scores for annotated clusters in the reference with clusters of known cell types. The use of a non-parametric statistic means that very high dynamic range of expression values are used in cell type annotation, which improves the results at least by virtue of significantly improved signal-to-noise and allows the methods to be extended to a great variety of cell, tissue, and sample types (compared to parametric statistics).

Systems and methods of the invention also include and provide other functions and benefits.

The system uses in silico tools for “cell calling”, i.e., distinguishing between partitions (e.g., droplets) that contain cells and background partitions that did not fully capture a cell. In addition, the system provides tools for “barcode processing” in which barcodes are optionally converted to a compact format and/or tested against a whitelist in a manner that greatly improves processing speed during deduplication while preserving information. Moreover, the system can implement tools for “multimapping” which preserved information for reads that map to multiple locations in a reference (which reads have previously typically just been discarded). Additionally the system includes analytical tools for analytics, e.g., clustering, display, classification, and metrics extraction, e.g., useful to show what clusters of cells—based on gene expression patterns—are present in the sample.

Those tools for cell calling, barcode processing, multimapping, and analytics are particularly well-suited to scRNA-Seq using particle-templated instant partitions (PIPs), which isolate a very large number of cells into emulsion droplets essentially simultaneously. To perform scRNA-Seq using PIPs, cells and particles are mixed together in an aqueous mixture. The particles may be hydrogel beads and those hydrogel beads may carry reagents such as enzymes and/or oligonucleotides. The reaction mixture may be provided with any of a variety of reagents, either in the aqueous phase or provide on or within the hydrogel bead particles. Those reagents may include, besides any enzymes or oligonucleotides, dNTPs, co-factors, dyes, salts, chemicals, others, or combinations thereof. The aqueous mixture will typically be provided with a number of hydrogel bead particles that corresponds to a number of aqueous partitions—aka droplets or “PIPs”—to be made. The cells may be introduced at a concentration, i.e., a dilution, such that PIPs will be made with an average number of cells per PIP being no greater than one. With the aqueous mixture prepared, an oil may be added over the aqueous phase (optionally while providing the mixture with a surfactant). The oil/aqueous mixture may then be sheared, e.g., by vortexing the tube (such as an 0.5 mL microcentrifuge tube or larger conical tube). The shearing energy causes each of the hydrogel bead particles to function as a template, nearly-instantly causing a partition of the aqueous liquid to be surrounded by the oil, forming a plurality of stable monodisperse emulsion droplets, aka partitions, hence particle-templated instant partitions (PIPs). An arbitrarily large number of PIPs may be formed simultaneously in one reaction volume and nearly instantly during vortexing.

Each PIP includes, with very few exceptions, one particle, at most one cells, cell lysis reagents (e.g., such as a proteinase enzyme), oligos for RNA hybrid capture, such as barcoded poly-T capture oligos packed within or covalently linked to the particles. The PIPs may further include other reagents, such as reverse transcriptase, dNTPs, other capture oligos (e.g., template-switching oligos) either linked to the particles or free in solution, primers, co-factors, etc. When the partitions, or PIPs, form instantly, each will contain-on average-one or zero cells. The tube may be transferred to a thermocycler or other temperature control device. Using the provided reagents, the cells may be lysed to release RNA and the RNA may be captured to the oligos (commonly by hybrid capture by also optionally by enzymatic attachment, e.g., by a ligase or transposase). Typically, all of the oligos of one hydrogel bead particle share copies of a common “cellular barcode” that is (essentially) unique to that particle. Additionally, each oligo may include a UMI which is unique (or at least nearly-unique or essentially or effectively unique to that oligo) at least among the oligos of that particle. After the RNA molecules are captured to the oligos, typical RNA-Seq will proceed by cDNA synthesis, amplification and ligation of sequencing adaptors, and sequencing to produce sequence data that includes nucleotide sequences from the RNA molecules and from the oligos (e.g., from the barcodes). Described herein are systems for barcode processing, cell calling, multimapping, and analytics using that sequence data.

Barcode processing according to systems of the invention may use a tiered barcode system, optionally every tier includes one of a specified list of possible barcodes. Tier may refer to a level of information specific to a barcode, such as a cellular barcode then a UMI. There may also be higher-level barcodes, such as for different patients or samples, and/or intermediate level barcodes, such as barcodes specific to genes (e.g., if sequence-specific primers may be used). Typically scRNA-Seq will include cell and UMI barcodes, but other tiers may be included for any category of information, such as date or location. Including barcodes in the reagent oligonucleotides leads to the sequence data include the sequence from the barcodes. A computer system can match the barcodes against a list or database, to assign sequence reads to cells or individual molecules. Barcode matching may be done highly efficiently by matching each tier in isolation. Some embodiments of computer systems of the invention allow a Hamming distance of 1. Limiting the Hamming distance to 1 means that the bases in a sequence read, e.g., “read 1” (“R1”) within a FASTQ file that correspond to each tier's position can differ from a barcode by one base and still be matched to that barcode. To simplify and reduce the size of the barcode list sent to a reference-mapping module such as STAR, the tiered barcodes may be replaced by a new set of generated barcodes. For example, each barcode may be replaced by a number such as its numerical position on a list of barcodes. In some embodiments, new barcodes are generated only for the barcodes with at least one matching read. That is, if trillions of barcodes are calculated to have formed the inputs to an experiment, and the system processes the resulting FASTQ files and identifies only billions of barcodes in reads, that new, limited list of barcodes may be assigned new barcodes with significant savings in computational resources downstream. The methods of barcode processing aid in keeping downstream steps computationally tractable while maintaining the ability to provide a measure of expression levels when mapping reads to a reference and using UMI counts to de-duplicate reads.

The use of PIPs (where a large number of cells are isolated into aqueous partitions simultaneously) compared to microfluidic platforms (where a limited number of cells are isolated into partitions serially) brings to light a number of challenges associated with the throughput and data volumes first met by PIPs. For example, using PIPs reveals that it would be beneficial to have automated and accurate tools for correctly calling whether a partitions contains a cell, as opposed to being a background partition that did not fully capture a cell, aka cell calling. The disclosure recognizes that, as a practical matter, at the data volumes and throughput associated with PIPs, distinguishing partitions that contain cells from background partitions (that did not fully capture a cell) can be done automatically. For example, from the sequence data, all of the barcodes that are identified may be ranked in order by the number of UMIs associated with each barcode. Barcodes associated with a relatively high number of UMIs are more likely to arise from cells than from background partitions, while barcodes associated with a relatively low number of UMIs are more likely to arise from background partitions. Systems of the invention may present the user with a choice of sensitivity/specificity (e.g., choose one of five pre-set options). Behind the scenes, a computer system can calculate the number of UMIs (e.g., on y-axis) over rank of ranked barcodes (x-axis), fit a function to the curve, take derivatives to find inflection points, and populate a user interface with chose from those points along the curve. The user may select the sensitivity level for cell calling and the analysis proceeds with the selected sensitivity. For example, a user seeking to classify cell types that are abundantly present in a tissue sample, may choose a low sensitivity (willing to discard some cell data in favor of excluding substantially all background partitions). A user interested in very rare cells, e.g., cancer cells, among a sample of blood cells may select very high sensitivity. The computer system can present guidance to the user on selecting sensitivity. After any cell calling steps, the system perform barcode processing.

The present invention uses multimapping to preserve information when a sequence read maps to more than one location in a reference. The sequencing steps of RNA-Seq typically provide a plurality of sequence reads, often in a FASTQ format. Analysis of those reads typically involves mapping each read to a reference, such as a mRNA or gene sequence listing used specifically for expression analysis or even simply the published human genome. Mapping reads to a reference commonly includes some version of an alignment algorithm such as, one or any combination of, pairwise alignment, alignment of a Burrows-Wheeler transform, comparing k-mers of a string to hashed k-mers of the reference, or alignment using a suffix or prefix tree built from the reference and/or the reads. Various tools are available in the art for performing alignments according to such methods (see, e.g., Dobin, 2013, STAR: ultrafast universal RNA-seq aligner, Bioinformatics 29(1):15-21, incorporated by reference). An alignment finds a best-matching location within a reference for each read and, by implication, the gene (represented in the reference) from which an RNA transcript (represented in the read) was produced. There are a number of reasons rooted in biology and informatics why a read may map to a reference in more than one location. For example, the reference may include homologous genes (e.g., from duplications) or copy number variations, or the read may be short enough that it admits of two or more distinct mappings to the reference. Prior art methods have simply discarded such ambiguously mapping reads. In contrast, systems of the invention treat such results as a multiple mapping of the read and preserve the read as a multimapped read. Preserving information about multimapped reads allows scRNA-Seq sequencing data to be analyzed to discover biological information about the expression of homologous genes or copy number variation in a sample.

After barcode processing, cell calling, and read-mapping, systems of the invention provide tools for analytics of results of scRNA-Seq data. Analytics according to the invention generally includes clustering of cells, differential expression analysis, and extraction of various metrics to provide among other things, for example, a measure of expression levels of various genes in each cells. Systems of the invention provide a variety of tools to aid in display, review, and interpretation. For example, the clustering methods may provide for classification of cells. Among other things, systems of the invention may be used to output basic metrics, a barcode rank plot, a clustering map, a differential expression table, or combinations thereof. Preferred embodiments of the system operate in a computer system that includes at least one processor coupled to a memory subsystem. The functionality provided by the system may be implemented in a local computer, a server system, or a cloud-based computing system. Software modules to perform the described functionality may be developed in any suitable environment such as, for example, python, Ruby on rails, c++, others, or combinations thereof. Versions of systems of the invention are executable in mac osx, Linux, and windows operating systems. Preferred embodiments operate in server or cloud environments and are operable to receive sequence data, e.g., in a FASTQ or FASTA format, from a sequencing resource such as a next-generation sequencing (NGS) instrument or a genomics facility. The system may align reads to a reference generating a sequence alignment map (SAM) or binary alignment map (BAM) and provide outputs to a user computer. Details and variations of embodiments of functionality of the system are described herein.

In certain aspects, the invention provides a system for expression analysis with cell calling. Systems include a processor coupled to a memory subsystem comprising instructions executable by the processor to cause the systems to: receive sequence data produced by sequencing RNA from a plurality of partitions; correlate, for each partition, counts of barcode sequences in the sequence data to a probability that the partition contained one entire isolated cell; receive a user selection for a sensitivity for the probability that the partition contained one entire isolated cell; and analyze mRNA levels among the partitions that meet the user-selected sensitivity.

The system may be operable to present (via a graphical user interface displayed on a computing device with an input/output interface) the user with at least three (e.g., 5) distinct calculated sensitivity levels and receive the users selection via the computing device. In some embodiments, the system correlates the barcode sequences to the probability by calculating a function of barcode count over barcode rank, dividing the function by a pre-determined value, and selecting a cutoff level for barcode count, above which a partition is deemed to contain one entire isolated cell. The system may be operable to, after analyzing the mRNA levels, re-analyze mRNA levels with a new user selection for the sensitivity. In certain embodiments, analyzing the mRNA levels includes assigning sequence reads to cells using a cellular barcode, deduplicating sequence reads using a universal molecular identifier, mapping deduplicated reads to a reference, and counting deduplicated reads that map to a gene in the reference as a measure of expression level of that gene. The system may be operable to downsample the sequence data by mapping to the reference fewer than all of the deduplicated reads. In some embodiments, the number of the deduplicated reads that is mapped is selected so that the mRNA level from the sequence data will be normalized to levels calculated from at least one other experiment.

Aspects of the invention provide systems for expression analysis with multimapping. Such a system includes a processor coupled to a memory subsystem comprising instructions executable by the processor to cause the system to: receive sequence data (e.g., Illumina output) produced by sequencing RNA from a single-cell; map at least one sequence read from the sequence data to a reference comprising reference gene information; identify at least a first location and a second location in the reference where the sequence read maps with at least a threshold matching score; store the sequence read in the memory subsystem with markup identifying the read as mapping to at least the first or the second location in the reference. Preferably the system is operable to map a plurality of reads to the reference and select the first or the second location in the reference for the sequence read based on the location to which a greater number of the plurality of reads map. The system may be operable to store the sequence read with markup identifying the read as mapping to both the first and the second location. In some embodiments, the system assigns a first weight to the mapping to the first location and a second weight to the mapping to the second location. The first and second weight may each be based at least in part on a respective first and second alignment score between the sequence read and the reference. The system may be operable to use the markup and the reference gene information to provide a report describing a gene duplication or copy number variation in the single cell.

In some aspects, the invention provides systems for expression analysis with barcode processing. Such a system includes a processor coupled to a memory subsystem comprising instructions executable by the processor to cause the system to: receive sequence data produced by sequencing RNA from a single-cell; compare barcodes from sequence reads in the sequence data to a barcode whitelist; when a barcode in one sequence read fails to match a whitelist barcode with a predetermined Hamming distance value, omit the one sequence read from further analysis, wherein for each sequence read, a barcode from a first tier is compared to a cellular barcode whitelist and a barcode from a second tier is compared to a UMI whitelist; deduplicate reads for which first tier and second tier barcodes match the whitelist within the predetermined Hamming distance value; and provide a measure of mRNA levels from the deduplicated reads. The system may be operable to replace the barcodes in the sequence reads with index values that occupy less space in the memory subsystem prior to the deduplication step.

For each of the foregoing aspects, the invention provides corresponding methods that include performing the recited functions using a system as described. Thus, the invention provides systems and methods for evaluating gene expression levels from scRNA-Seq experiments. The systems and methods use in silico cell calling tools for distinguishing between partitions that contain cells and background partitions that did not fully capture a cell. The systems and methods also provide barcode processing tools that tests barcodes from at least cellular and UMI tiers against a whitelist and optionally converts those to a compact index format. Further, the systems and methods can implement multimapping tools to preserve information for reads that map to multiple locations in a reference.

### BRIEF DESCRIPTION OF THE DRAWINGS

FIG. 1 shows a barcode rank plot.

FIG. 2 shows a barcode rank plot for HEK/3T3 cells.

FIG. 3 shows a barcode rank plot for peripheral blood monocytes (PBMC).

FIG. 4 shows a barcode rank plot for breast tissue sample.

FIG. 5 shows cell calling at five sensitivity levels for a PBMC sample.

FIG. 6 shows a clustering result for an HEK/3T3 sample.

FIG. 7 shows a clustering result for a PBMC sample.

FIG. 8 shows cell clustering.

FIG. 9 shows correlation of U for cluster 7 to a B cell reference cluster U.

FIG. 10 shows correlation of U for cluster 7 to a CD4 T cell reference cluster U.

FIG. 11 shows annotation of cell clusters.

FIG. 12 is a barnyard plot that shows numbers of transcripts by organism type.

FIG. 13 shows a system of the invention.

### DETAILED DESCRIPTION

The present disclosure provides a software platform that analyzes single-cell RNA data that may be obtained using particle-templated instant partitions (PIPs). Systems of the invention offer a comprehensive analysis solution that provides the user with detailed metrics, gene expression profiles, and basic cell quality and clustering indicators. The outputs of the system may also be used for subsequent, specialized tertiary analysis streams.

### System Requirements

Preferred embodiments of the system may run on Windows, macOS and Linux operating systems. Preferably the system includes at least about 64 GB of RAM and 1 TB hard disk space (to accommodate multiple results). In some embodiments, a version of the system running on Windows uses a local installation of Docker or similar, which can allow the system to seamlessly interoperate with modules that are not themselves compatible with Windows.

### Pipeline Overview

Systems of the invention may perform any combination of the following steps:
1. FASTQ processing, including barcode matching, QC and (optionally) down-sampling
2. Mapping
3. UMI counting
4. Cell calling
5. Clustering
6. Differential expression
7. Cell type annotation
8. Metrics extraction
9. Report generation

### 1. Barcode Processing

The sequencing process of scRNA-Seq produces a plurality of sequence reads, typically in one or more FASTQ files. One benefit of PIPs (over legacy microfluidics) is that large numbers of cells may be simultaneously isolated in partitions and then interrogated to produce a single FASTQ file (without the need to concatenate FASTQ files from different runs/days). Processing may begin with processing the FASTQ files to: extract the reads that are associated with a barcode; remove unwanted technical sequences from the data before mapping; and optionally to down-sample the data. Modules of the system, e.g., optionally developed in python, C++, or other such environment, may read in from a FASTQ file and process each entry to read barcode sequence and RNA sequence. Typically, the barcode sequence will be compared to a list of barcodes and the RNA sequence data will be mapped to a reference to identify the gene or transcript.

In certain embodiments, barcode processing according to systems of the invention may use a tiered barcode system, in which optionally every tier includes one of a specified list of possible barcodes. Tier may refer to a level of information specific to a barcode, such as a cellular barcode then a UMI. There may also be higher-level barcodes, such as for different patients or samples, and/or intermediate level barcodes, such as barcodes specific to genes (e.g., if sequence-specific primers may be used). Typically scRNA-Seq will include cell and UMI barcodes, but other tiers may be included for any category of information, such as date or location. Including barcodes in the reagent oligonucleotides leads to the sequence data that include the sequence from the barcodes. A computer system can match the barcodes against a list or database, to assign sequence reads to cells or individual molecules. Barcode matching may be done highly efficiently by matching each tier in isolation. Some embodiments of systems of the invention allow a Hamming distance of 1. Limiting the Hamming distance to 1 means that the bases in a sequence read, e.g., “read 1” (“R1”) within a FASTQ file that correspond to each tier's position can differ from a barcode by one base and still be matched to that barcode.

To simplify and reduce the size of the barcode list sent to a reference-mapping module such as STAR, the tiered barcodes may be replaced by a new set of generated barcodes. For example, each barcode may be replaced by a number such as its numerical position on a list of barcodes. In some embodiments, new barcodes are generated only for the barcodes with at least one matching read.

### Quality Control

In some embodiments, an R2 (Read 2) fastq file includes the cDNA construct and also includes technical sequences related to the PIP chemistry, for example, the template switch oligo (TSO) and poly-A sequences. The prevalence of those sequences may be inversely correlated with the fragment size. Because a read is less likely to be mapped if it contains extraneous sequences, it may be preferable to remove the TSO sequences from the 5′ end and poly-A sequences from the 3′ end in the R2 fastq file. In addition, depending on chemistry type, a fixed number of non-cDNA bases may be removed from the 5′ end. Reads that are less than 20 bases long after trimming may be discarded from further analysis.

After all reads are matched to barcodes, inevitably some barcodes will be associated with a small number of reads. Those barcodes are highly unlikely to be classified as cells later on, so it may be economical to exclude them and the associated reads from further analysis. The user can define a minimum number of reads per barcode. If such a threshold is specified, reads associated with low count barcodes will not be passed on to STAR for mapping. See “cell calling” discussed in greater detail elsewhere herein.

### Downsampling

In some experiments it might be desirable to standardize sequencing depth, so that experimental conditions could be reliably compared. Therefore, systems of the invention are operable to downsample data to a specified number of reads. Reads may be selected randomly, with the probability of selection determined by the total number of reads (provided by the user or determined by counting the lines in the fastq inputs).

### Outputs

In certain embodiments, the fastq processing step results in the following outputs:

- <output-root>/metrics/barcode_stats. json: General
statistics about the quality of barcode matching.
- <output-
root>/metrics/barcodes/Generated_barcode_read_info_table: A list
of all the generated barcodes that are passed on to STAR for
mapping and counting. The list includes the tiers associated
with the original barcodes, and the number of perfect and error-
corrected matches for each barcode.
- <output-root>/metrics/barcodes/barcode_whitelist: A list of
all the generated barcodes provided to STAR as a “whitelist” for
mapping and counting.
- <output-root>/barcodes_fastqs/*: Intermediate fastq files
to be used by STAR for mapping. Unless instructed otherwise,
these files are deleted at the end of the analysis.

### 2. Mapping

The sequencing steps of RNA-Seq typically provide a plurality of sequence reads, often in a FASTQ format. Analysis of those reads typically involves mapping each read to a reference, such as a mRNA or gene sequence listing used specifically for expression analysis or even simply the published human genome. Mapping reads to a reference commonly includes some version of an alignment algorithm such as, one or any combination of, pairwise alignment, alignment of a Burrows-Wheeler transform, comparing k-mers of a string to hashed k-mers of the reference, or alignment using a suffix or prefix tree built from the reference and/or the reads. Various tools are available in the art for performing alignments according to such methods. Certain preferred embodiments use a read-mapping module referred to as STAR. See Dobin, 2013, STAR: ultrafast universal RNA-seq aligner, Bioinformatics 29(1):15-21, incorporated by reference. Specifically, systems of the invention use STAR (Spliced Transcripts Alignment to a Reference) and the associated STARsolo to map reads to a transcriptome and quantify transcript abundance. STAR uses a special index to be built from the original transcriptome of interest. Some common indices, such as human and mouse, can be downloaded from sources such as Fluent BioSciences. However, for some unique situations, systems of the invention allow a user to create a unique, or experiment-specific, index.

Documentation for the STAR products are available from the publisher. In preferred embodiments, systems of invention store the output of read-mapping (e.g., by STAR) in a directory such as <output-root>/starsolo e.g., as described in the STAR documentation.

It is noted that to the extent that STAR is not yet supported on Windows and Mac, the system will run STAR using a Docker image. When running in those operating systems (or if explicitly requested in Linux), systems of the invention will launch STAR from a Docker image.

Read mapping generally refers to detecting a location within a reference to which a sequence read best maps. They system includes programming logic for using information that is potentially revealed when a sequence read maps well to more than one location on a reference.

The present invention uses multimapping to preserve information when a sequence read maps to more than one location in a reference. There are a number of reasons rooted in biology and informatics why a read may map to a reference in more than one location. For example, the reference may include homologous genes (e.g., from duplications) or copy number variations, or the read may be short enough that it admits of two or more distinct mappings to the reference. Prior art methods have simply discarded such ambiguously mapping reads. In contrast, systems of the invention treat such results as a multiple mapping of the read and preserve the read as a multimapped read. Preserving information about multimapped reads allows scRNA-Seq sequencing data to be analyzed to discover biological information about the expression of homologous genes or copy number variation in a sample. Importantly, reads that multimap are preferably not discarded (compared to prior art systems that discard such reads). In some embodiment, the read is assigned to one of the locations according to a selection logic such as by random, or (where there are two locations) by alternating assignments between the two locations as multiple reads map to the two, or by mapping each read to the location to which majority of reads map, or by giving “partial credit” (e.g., pro rata over the number of different locations, or weighted by an alignment score), or others, or combinations thereof.

### 3. UMI Counting

Methods may use two oligonucleotide barcodes that get added to RNA molecules or cDNA copies thereof. The sequences of those two barcodes appear in sequence data obtained from the cellular RNA. Typically, one of those barcodes is the same throughout a droplet but different among the droplets, and functions as a cellular barcode that provides a common cell-identifying tag for all molecules from one cell. Using a cellular barcode allows for pooling nucleic acids from multiple cells during sequencing and subsequent separation of sequence data by cell in silico. The other barcode often used in scRNA-Seq is referred to as a unique molecular identifier (UMI). A UMI tags each individual molecule with a unique sequence of nucleic acid bases. When those molecules are amplified, e.g., by PCR, amplicons from one molecule share a common UMI sequence. After sequencing, sequence reads that contain identical genetic sequence data and UMI sequence data can be treated as duplicates. In process sometimes referred to as de-duplicating reads, all identical reads are treated as one and, after de-duplication, unique reads are counted. A UMI need not be unique and may be “nearly unique”, meaning that a small number of duplications do not negate the value of the experiment. What is important is that statistically the number of duplicate barcodes is not significant. The count of unique reads from a gene is a count of a number of mRNA transcripts from that gene that were present in the cell. The numbers of mRNA transcripts from various genes in a cell provides a measure of gene expression levels for that cell.

After STAR is done mapping the reads in the fastq files to the reference, it creates a .bam file (a binary alignment map (BAM), which is binary version of the sequence alignment map, or SAM, file). The system then parses the BAM file to create a dataset with all the molecules (barcode+UMI combinations) in the sample that were mapped to the reference. The molecule info is saved, e.g., to an HDF5 format file with a suitable name such as molecule_info.h5. The file may be analyzed by the system to create a raw count (feature-barcode) matrix. In some embodiments, the matrix is contained in the files: matrix.mtx.gz, barcodes.tsv.gz and features.tsv.gz, e.g., all in a directory such as raw_matrix. Those files form a sparse matrix containing a full count of unique UMIs for each barcode and gene. The format of the matrix is preferably consistent with most downstream analysis tools, including third-party analysis tools.

### 4. Cell Calling

After obtaining the UMI count matrix, barcodes are separated into cell-containing PIPs and background PIPs, i.e. PIPs that did not fully capture a cell. This separation is based on the barcode rank plot, which orders barcodes based on the number of unique UMIs associated with them. The use of PIPs (where a large number of cells are isolated into aqueous partitions simultaneously) compared to microfluidic platforms (where a limited number of cells are isolated into partitions serially) brings to light a number of challenges, first met by PIPs, associated with throughput and data volumes. For example, using PIPs reveals that it would be beneficial to have automated and accurate tools for correctly calling whether a partitions contains a cell, as opposed to being a background partition that did not fully capture a cell, aka cell calling. The disclosure recognizes that, as a practical matter, at the data volumes and throughput associated with PIPs, distinguishing partitions that contain cells from background partitions (that did not fully capture a cell) would benefit from being done automatically, even “on-the-fly”. For example, from the sequence data, all of the barcodes that are identified may be ranked in order by the number of UMIs associated with each barcode. Barcodes associated with a higher number of UMIs are more likely to arise from cells while barcodes associated with a lower number of UMIs are more likely to arise from background partitions.

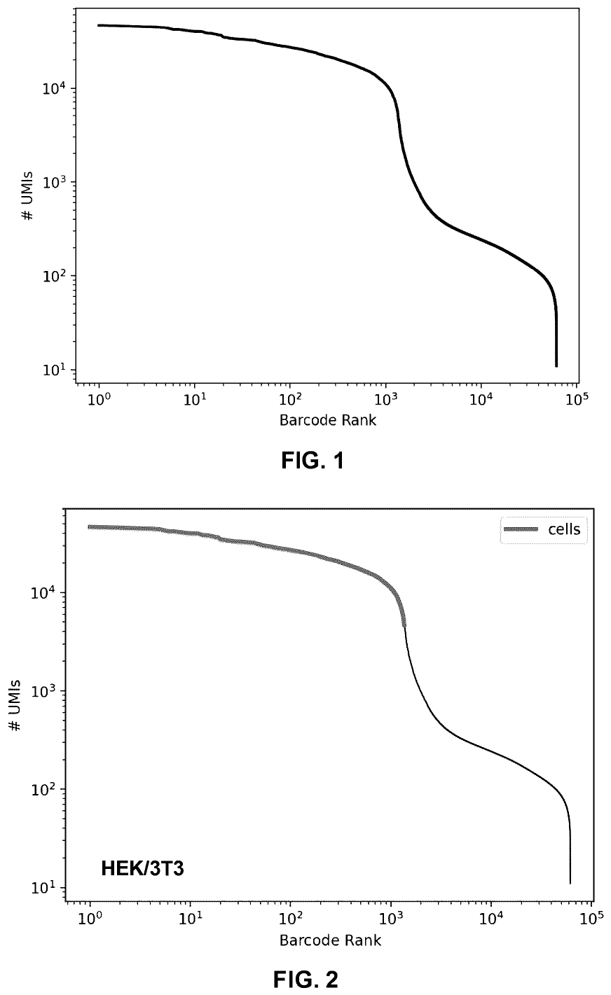

FIG. 1 shows a barcode rank plot. Typically, the cell barcodes are concentrated at the top mode of the rank plot, whereas the background barcodes are concentrated in the low UMI count mode. Therefore, the purpose of cell calling is to find a point in the first “knee” area that separates the two modes. One approach for finding that point involves dividing the number of UMIs at the top of the rank plot by a constant denominator (e.g., 10). Methods of finding the inflection point may be highly sensitive to the shape of the rank plot, which varies strongly between different sample types. Systems of the invention may automatically find a point on the barcode rank plot that divides cell partitions from background partitions, or systems may present users with one or more options aiding in allowing the user to set or select that point, or system may perform a combination of automatic selection or suggestion in combination with user selection.

Systems of the invention may present the user with a choice of sensitivity/specificity (e.g., choose one of five pre-set options). Here plots are given to show the result of calling cell barcodes up to 1/10 of the top number of barcodes for HEK/3T3, PBMC and breast tissue samples.

FIG. 2 shows a barcode rank plot for HEK/3T3 cells.

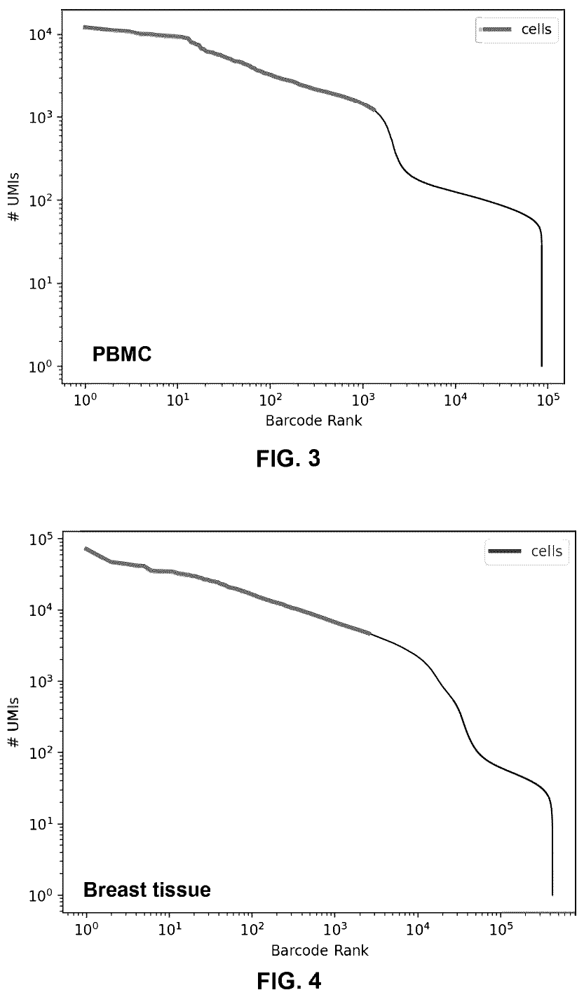

FIG. 3 shows a barcode rank plot for peripheral blood monocytes (PBMC).

FIG. 4 shows a barcode rank plot for breast tissue sample.

The cell portion in each is highlighted in bold. Cell calling for HEK/3T3 seems consistent with a visual inspection of the barcode rank plot: the entire high-UMI mode is captured. In the cases of PBMCs and breast cells, however, the number of cell barcodes is clearly underestimated, because the top mode has a more negative slope and is less clearly defined. This may be a selection made deliberately by the user. For example, compared to HEK/3T3, a user studying PBMCs may, due to underlying biological reasons, need to optimize the assay for data volume (generously ignoring or excluding background PIPs) due the qualities of information being sought.

In some embodiments, to accommodate for differences between samples and facilitate accurate cell calling, systems of the invention provide cell calling at numerous (e.g., five) different sensitivity levels using the following steps:
1) Select a starting point, S, close to the top of the rank plot. To avoid selecting an outlier, the system uses the point where the slope of the plot is below 10% for the first time as a starting point.
2) Let M be the number of UMIs for the barcode at the starting point. For each cell calling sensitivity level L, find a number of UMIs, U, such that:

$${U}_{i} = M * {10}^{- ( 0.5 + 0.25 {L}_{i} )} , i = \{ 1 , 2 , 3 , 4 , 5 \}$$

3) For each cell calling sensitivity level Li, all barcodes with a UMI count higher than or equal to Ui are selected as cell-containing barcodes.

A system may use any suitable approach to determining a spot that includes a knee or an inflection point. For example, behind the scenes (or on-screen, e.g., shown to a user graphically), a computer system can calculate the number of UMIs (e.g., on y-axis) over rank of ranked barcodes (x-axis), fit a function to the curve, take derivatives to find inflection points, and populate a user interface with options for points chosen along the curve. The user may select the sensitivity level for cell calling and the analysis proceeds with the selected sensitivity. For example, a user seeking to classify cell types that are abundantly present in a tissue sample, may choose a low sensitivity (willing to discard some cell data in favor of excluding substantially all background partitions). A user interested in very rare cells, e.g., cancer cells, among a sample of blood cells may select very high sensitivity. The computer system can present guidance to the user on selecting sensitivity.

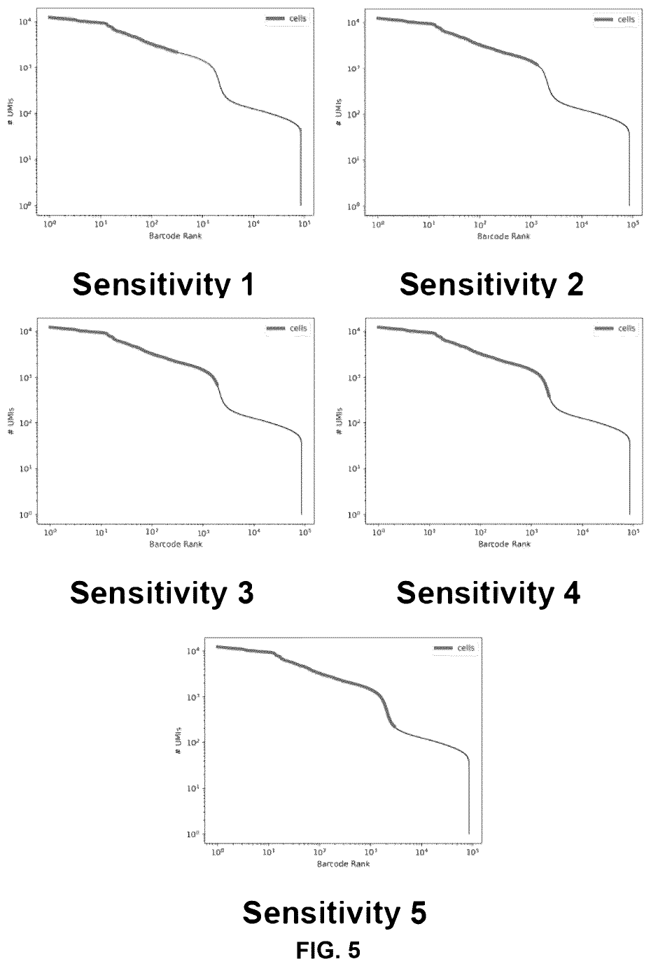

FIG. 5 shows cell calling at five sensitivity levels for a PBMC sample.

Cell calling, by its nature, involves a tradeoff between the number of cells and their quality. One can arbitrarily increase the number of cells by calling deeper into the rank plot, but this increases the likelihood of selecting low-RNA content or otherwise compromised cells. The optimal number of cells recovered depends on the sample type and purpose of each experiment. For example, if some cell types in a sample (such as neutrophils or red blood cells) have low RNA expression, they may be present in high numbers in the lower UMI region of the plot, or the UMI count distribution could even be unimodal. In that case, high-sensitivity cell calling may be warranted. On the other hand, if high clustering accuracy is required for precise cell type characterization, it would be preferable to use low-sensitivity cell calling. Cell calling at different sensitivity levels allows users to observe a wide range of possible outcomes and determine the correct sensitivity for their data.

### Manual Selection of Number of Cells

In some cases users may want more precise control of the number of called cells, beyond what is afforded with the five sensitivity levels. In some embodiments, the system allows users to specify a desired number of cells, in which case this exact number of barcodes is selected from the top of the rank plot. The outputs described herein will be generated for the selected set of barcodes. For some embodiments of systems of the invention, this may be executed from the command line, e.g., using the prefix force_N, with N being the number of cells input by the user.

### Outputs

After cells are called, a directory may be created for each sensitivity level inside a parent directory such as:

<output-root>/cell_calling, containing a text file listing the indices (starting at 1) of the barcodes selected as cells and an image with a barcode rank plot. Additionally, a filtered count matrix may be generated for each sensitivity level and can be used for downstream analysis. Filtered matrices are stored in <output-root>/cr_filtered_quant. The skilled artisan will understand how to write the functionality to save those outputs in a suitable programming or development environment such as python.

Beyond barcode processing, cell calling, and read-mapping, systems of the invention provide tools for analytics of results of scRNA-Seq data. Analytics according to the invention generally includes clustering of cells, differential expression analysis, and extraction of various metrics to provide among other things, for example, a measure of expression levels of various genes in each cells. Systems of the invention provide a variety of tools to aid in display, review, and interpretation. For example, the clustering methods may provide tools useful for classification of cells.

### 5. Clustering

After obtaining gene expression data, methods of the invention are used to conduct a clustering analysis to identify or define clusters of cells of the same type. Methods may include quantifying the plurality of distinct mRNA molecules and optionally generating an expression profile for the cells after quantifying the plurality of distinct mRNA molecules. Prior to clustering, it may be beneficial to perform certain pre-processing steps. Specifically, expression data for the cells may be pre-processed using one or combination of (i) normalization; (ii) log transformation; (iii) gene selection; and (iv) scaling.

The starting point for clustering is preferably a sparse matrix representation of the filtered count matrix, in which every row represents a cell and every column represents a gene. Any combination of the pre-processing steps may be performed.

Cell normalization methods may be applied to (i) normalize expression levels within and for each cell (improving inter-cell comparisons). Each cell's data (cells of each row of the matrix) are normalized to unit norm. This is performed so that cells with similar expression profiles but different relative RNA abundance can still be clustered together.

Log transform methods may continue to (ii) transform the resultant matrix by a function such as ln(x+1) to approximate a normal distribution.

Highly variable gene selection may be performed. At this stage, it may be preferable to (iii) select, from all of the genes, a subset of the genes with high variance (to greatly improve clustering). E.g., for every gene, the variance in expression across cells is calculated; the genes are then ranked by variance, and the top genes at the Nth percentile are selected. Selecting a subset of genes with high variance will maximize the efficiency of clustering. For every gene, the variance in expression across cells is calculated. The genes are then ranked by variance, and the top genes at the Nth percentile are selected, with N being a user input.

Scaling may be performed. At this stage, it may be preferable to (iv) scale the feature columns (genes) so that each column has a mean of 0 and a standard deviation of 1.

Systems of the invention may use Principal Component Analysis (PCA). The optionally pre-processed gene expression data may be subject to principal component analysis (PCA), to reduce the dimensionality of data to a limited (e.g., 2, 3, 4 . . . ) number of principal components that best account for variance in the data. Performing PCA provides a PCA matrix, a matrix in which cells are positioned by gene expression level such that similar cells are more proximal to each other than to dissimilar cells. The PCA matrix may be subject to an automated process for defining clusters of similar cells.

That is, “clustering” may be performed.

Any suitable process may be used to identify clusters of similar cells from the PCT matrix (similar here refers to the real-world similarity of cells, meaning that cells will be grouped based cell type, e.g., all B cells with be together in a group that does not include natural killer cells). Suitable processes for identifying or defining cell clusters may include a community detection algorithm for example, or a method from the literature such as RaceID, SNN-Cliq, SINCERA, SEURAT, or SC3. Preferred embodiments involve constructing a k-nearest-neighbor (knn) graph from the PCA matrix and passing the graph to a Leiden algorithm to optimize cluster membership. The clustering process assigns the cells to clusters based on similarities among expression profiles.

In some embodiments, the system performs clustering using the Leiden algorithm, an art-recognized standard for graph-based clustering in scRNA-seq datasets. A k-nearest-neighbor (KNN) graph is constructed from the PCA matrix, which is converted to a directed node-edge graph and passed into Leiden. The Leiden algorithm uses a modularity optimization of graph communities to determine ideal cluster membership. Leiden has the added advantage of minimal user inputs (e.g., number of clusters, cutoffs, etc.) compared to other methods. See e.g., Franzen, 2020, Alona: a web server for single-cell RNA-seq analysis, Bioinformatics 36(12):3910-3912; Wu, 2020, Tools for the analysis of high-dimensional single-cell RNA sequencing data, Nat Rev Neph 16:408-421; and Zhu, 2020, Single-cell clustering based on shared nearest neighbor and graph partitioning, Interdiscip Sci 12(2): 117-130, all incorporated by reference.

In certain embodiments, the system also uses the popular K-means algorithm to divide cells into distributed clusters. It runs the algorithm for different values of K, representing a predetermined number of clusters. The minimum and maximum of the range of K values is controlled by the user and should be tailored to the number of cell types expected to be present in the sample.

### 6. Selection of Genes by Differential Expression

A challenging aspect of the annotation process is to select the subset of genes to include in the computation of similarity. Many genes are irrelevant because they are either not expressed at all or expressed similarly across cell types. Inclusion of additional irrelevant genes and exclusion of important genes will both decrease statistical power.

Current selection methods include using highly variable genes (HVGs): genes with variance across cells that are above a given threshold are included. That class of methods eliminates genes with no expression or very low expression and, to a lesser extent, genes with little variation compared to an expected model. Highly variable genes are not necessarily beneficial for cell type annotation because high variability can exist even between cells of the same type, especially with a zero-inflated count distribution seen in scRNA-seq data. Genes that are highly variable across the entire population of cells are not necessarily the ones that are useful in distinguishing cell clusters. Other selection methods look for fold change differential between groups: genes are chosen based on the relative difference between the average cell in a group and another group (one cluster or all other cells). That method is limited to the range of fold change values, which is limited. Genes with a high fold change are often differentially expressed, but do not capture the entire ensemble of genes that a statistical method would capture.

Certain embodiments involve gene selection by differential expression. For example, one method dubbed CIPR uses differentially expressed genes and the reference. See Ekiz, 2020, CIPR: a web-based R/shiny app and R package to annotate cell clusters in single cell RNA sequencing experiments, BMC Bioinformatics 21:a191, incorporated by reference. However, in the CIPR method, the downstream correlation step takes the matrix dot product of the log fold change values for the test and reference matrices, and does not employ a non-parametric statistical test score. Some methods use raw or normalized gene expression values to perform the correlation, but without a non-parametric statistical score, those values are susceptible to noise. Some genes have a baseline expression level orders of magnitude higher than other genes, and with a parameterized distribution, the variance in those highly expressed genes can easily overwhelm the difference between cell types in the expression of the lowly expressed genes. Using a non-parametric statistical score puts the genes on an equal playing field and has the added benefit of being a normal distribution with a high range of values.

For creating the reference dataset, instead of selecting variable genes across all cells, this method specifically selects genes that are most differentially expressed for each cell type, and therefore are expected to be more predictive of cell type.

Once clustering is completed, it may be of interest to determine the primary genes associated with each cluster. Some prior art methods require a parameterized probability distribution (e.g., the distribution or parameters of the distribution are specified). A parameterized probability distribution limits dynamic range or signal-to-noise ratio of the analysis. Here, gene expression data and, in particular, differential gene expression levels, are scored using a non-parametric statistical score such as the Mann-Whitney U score (or Wilcoxon rank sum), an Anderson-Darling test, or a bootstrap method. Using a non-parametric statistical score allows for a much greater dynamic range in differential gene expression levels, compared to fitting to a parameterized probability distribution. Expression profiles for each cluster are subject to a non-parametric statistical scoring, such as calculation of the Mann-Whitney U score, and correlations are calculated between the U scores for each clusters and U scores for annotated clusters in the reference with clusters of known cell types.

For this step, systems of the invention may use the Wilcoxon rank-sum test for independent variables (Mann-Whitney U). This nonparametric test is used to determine whether two samples come from the same distribution by ranking the values associated with two groups together, and calculating the sum of those ranks within each group. Those ranks are used to calculate a test statistic (U), based on which one can compare cluster and non-cluster cell populations. For each cluster, the system computes differential expression for genes associated with cells across that cluster vs. all other cells. The top genes, sorted by descending Z-score for each cluster, are exported to a csv file. This process is performed for different values of K in K-means clustering, as well as for graph-based Leiden clustering.

### 7. Cell Type Annotation

Cells are annotated with their cell types by a suitable method such as using the selected genes for each cluster in a comparison to a reference. Any suitable method may be used to annotate a cluster or its cells with cell type. For background, see Pasquini, 2021, Automated methods for cell type annotation on scRNA-seq data, Comp Struct Biotech J 19:961-969, Aran, 2019, Reference-based analysis of lung single-cell sequencing reveals a transitional profibrotic macrophage, Nature Immunol 20:163-172, Kiseley, 2018, scmap: projection of single-cell RNA-seq data across data sets, Nat Meth 15:359-362, de Kanter, 2019, CHETAH: a selective, hierarchical cell type identification method for single-cell RNA sequencing, Nucl Acids Res 47(16): e95, Hou, 2019, scMatch: a single-cell gene expression profile annotation tool using reference datasets, Bioinformatics 35(22):4688-4695, and Fu, 2020, clustifyr: an R package for automated single-cell RNA sequencing cluster classification, F1000Research 9:223, all incorporated by reference. Here, in preferred embodiments, each cluster will be compared to annotated clusters in a reference dataset. Based on the similarity to the reference dataset, each cluster gets assigned a primary cell type. Systems and methods of the invention are useful to perform automated cell type annotation using non-parametric statistical scores. Cell type for each cluster is determined by which cell type in the reference dataset has the highest similarity to that cluster based on a correlation value.

In certain embodiments, genes are selected to be included in the reference dataset based on their differential expression as discussed above. The reference dataset includes a union of the top N differentially expressed genes for each annotated cluster. The value of N can be a different value for different sample types, chosen to maximize correlation coefficients.

Instead of using gene expression values as an input to correlations, this method uses non-parametric statistical scores (such as U) describing how differentially genes are expressed in a cluster vs. all other clusters. This score (e.g., U) has a wider dynamic range of values, while also removing the noise in gene expression measurements. Examples of statistical tests that can yield such scores are the Wilcoxon ranked sum test (Mann-Whitney U statistic) or a t-test. In preferred embodiments, the cell or cluster annotation method uses statistical scores (such as the Wilcoxon ranked sum or Mann-Whitney U), but could use any pairwise statistical tests) as input to a correlation analysis between reference cell types and clusters, rather than the typical raw or normalized gene expression values. Differential gene expression is scored for clusters in the test data and the reference (e.g., the U score). Preferably, expression profiles for each cluster are subject to a non-parametric statistical scoring, such as calculation of the Mann-Whitney U score, and correlations are calculated between the U scores for each clusters and U scores for annotated clusters in the reference with clusters of known cell types. The use of such a score (e.g., “U”) is especially useful in cases with low numbers of cells or low amounts of material per cell, because the dynamic range of expression values is much smaller than the range of scores.

Any suitable method may be used to calculate a correlation between the U score for a test cluster and the U score for a reference cluster. Some methods use the Spearman (non-parametric) rather than the Pearson (parametric) correlation. In preferred embodiments of the disclosure, chosen genes follow a normal distribution, which allows for the calculation of a parametric correlation (e.g., Pearson), which increases the statistical power.

Regardless of the correlation calculation, differential expression-based gene selection and use of the non-parametric U scores drastically improves sensitivity (maximum correlation values) and signal-to-noise (differential between the maximum and next-highest correlation values) for individual cell types. The method can be generalized to any statistical test or cell types, from any single-cell technology, given the user provides a valid reference dataset.

Two innovations of the disclosure-the use of differential expression-based gene selection and non-parametric U scores-are manifest in the reference dataset, which includes non-parametrical statistical scores for each cell type/cluster and selected gene. Cell types in the reference correspond to clusters that were annotated manually or using an automated tool.

Briefly as an aside, the invention includes and provides methods for generating a reference from clustered scRNA data. Such methods may include (a) determining a cell type for each cluster manually (e.g., based on the presence of marker genes) or using an available annotation tool. Some clusters may be members of the same cell type; (b) performing differential expression analysis for each cell type; and (c) obtaining, for each cell type, the top N differentially expressed genes. The methods include (d) determining the union of all genes from the obtaining (c) step (noting that a gene can be differentially expressed in multiple cell types); (e) obtaining statistical score for each cell type and genes in the union obtained at (d); and (f) constructing a file containing the statistical scores obtained at (e), This is the reference dataset.

In the reference dataset, clusters are annotated with known cell types. Once the reference dataset is created or obtained, it may be used to annotate results from scRNA-seq experiments with cell type for cells in those experiments. Methods of the invention include obtaining gene expression data for a plurality of cells and assigning the cells to clusters based on the gene expression data, as discussed in “5. Clustering” above.

It is noted that the degree of certainty in annotation may be determined by the maximum correlation coefficient and by the difference between the maximum and second-highest correlation coefficients. Based on those two factors, in certain embodiments, methods of the invention make assignments within one of three categories: (i) definitive assignment, (ii) primary and secondary assignments, and (iii) no assignment. In such embodiments, if more than one cell type has high similarity, the second best-matching cell type gets assigned as a secondary cell type. The primary cell types are then used to generate a uniform manifold approximation and projection (UMAP) plot, e.g., colored by cell types. Clusters without high similarity to reference cell types are not assigned.

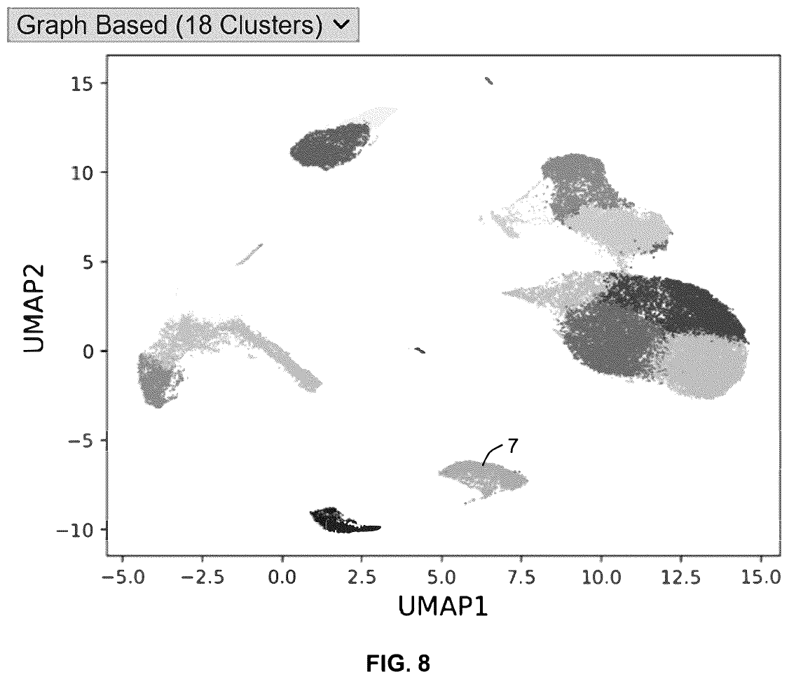

FIG. 8 shows cell clustering. Each cluster in FIG. 8 was found among the PCA matrix by k-nearest-neighbor graph creation and Leiden clustering. The depicted clusters are shaded differently. For each of those depicted clusters, methods of the invention are used for determining non-parametric statistical scores of differential gene expression for that cluster versus other clusters. Then, with those determined scores, methods of the invention proceed by calculating a correlation between the determined scores and annotated clusters in a reference. Here, the non-parametric score for the cluster labeled as cluster 7 in the test data in FIG. 8 will be correlated to each annotated cluster in a reference dataset. In the reference dataset, each cluster has been annotated as one of B cell, Naïve CD4 T cell, Memory CD4 T cell, central memory CD4 T, CD8 T, natural killer, CD14 monocyte, CD16 monocyte, dendritic, plasma, platelet, hematopoietic stem and progenitor cell (HSPC), and unknown. Preferably, a correlation is calculated between the U score for cluster 7 in FIG. 8 and the U score for each cluster in the reference dataset.

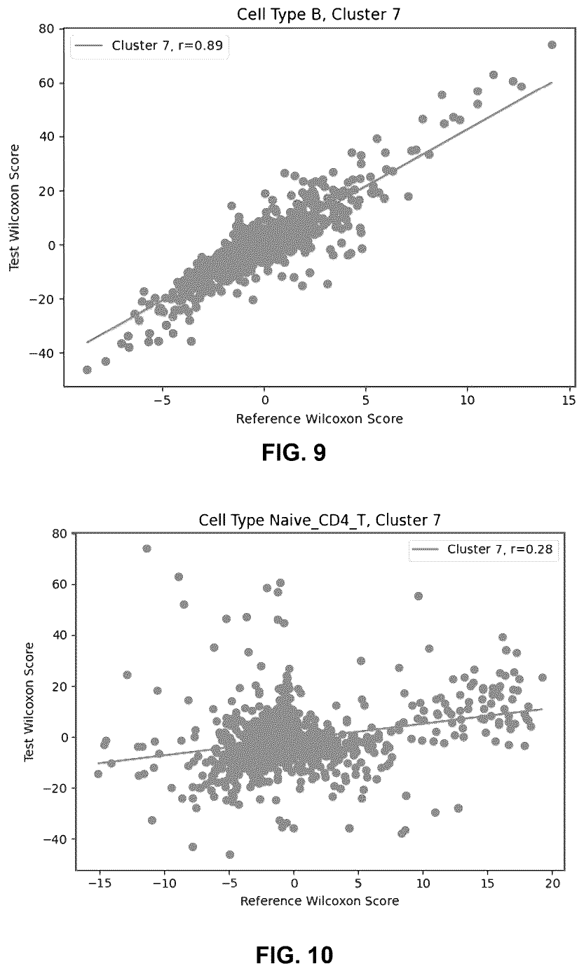

FIG. 9 shows correlation of U for cluster 7 to a B cell reference cluster U. In the depicted embodiment, the Wilcoxon score was used. The test U correlates to the reference U with an r value of 0.89, supporting that cluster 7 in FIG. 8 includes B cells. FIG. 9 shows that, for cluster 7 in the example dataset, the B cell type had a high correlation among the selected genes.

FIG. 10 shows correlation of U for cluster 7 to a CD4 T cell reference cluster U. As shown, the U score for cluster 7 correlates to Naïve CD4 T cells with an r value of 0.28, indicating that it is much less probable that cluster 7 includes naïve CD4 T cells. FIG. 10 shows that cluster 7 and the cell type with the second highest correlation gave a much lower correlation. These correlations are calculated for each cluster in FIG. 8, and each cluster is assigned an annotation based on the highest correlation.

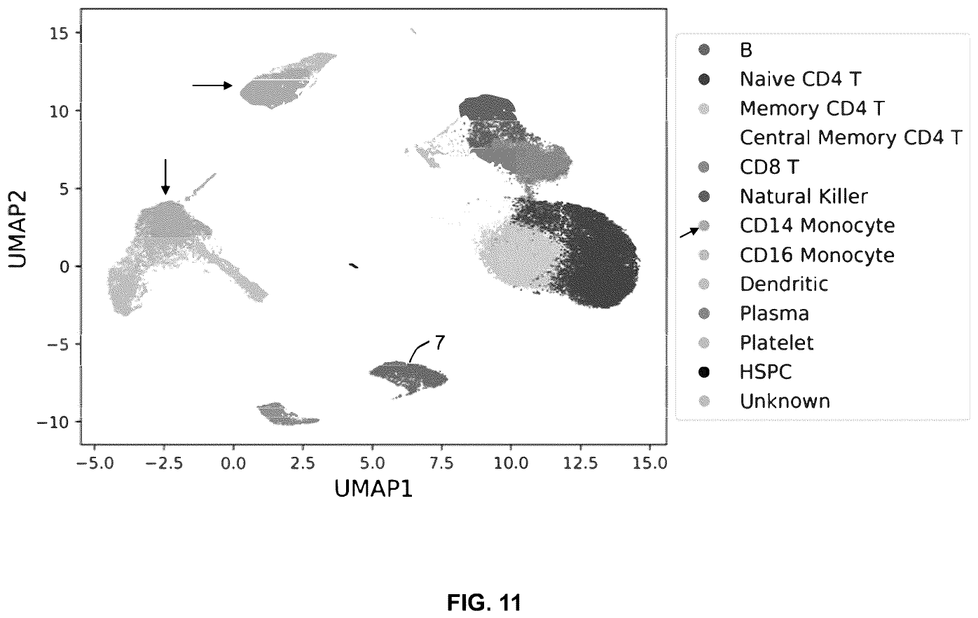

FIG. 11 shows cell type annotation by cluster, with unassigned clusters colored in neutral gray. The figure presents graph-based clustering results for a PBMC sample. It can be seen from the key that cluster 7 is identified as including B cells. It can be seen that, for example, CD8T cells and natural killers are adjacent to each other (abutting, even), but distinctly called. Two small arrows are drawn on FIG. 8 and at one place on the key to show that in some cases, more than on cluster will get assigned the same cell type (here, CD14 monocytes). It is also noted that some clusters do not get assigned and are given only neutral shading. The figure shows that, through the use of non-parametric scores of differentially expressed genes as inputs to a correlation between test and reference clusters, an annotated reference dataset may be used to annotate clusters, and cells included in those clusters. Those results were generated by the high-throughput, massively parallel technique of droplet-based scRNA-seq that makes use of pre-templated instant partitions (PIPs).

### Outputs

Among other things, systems of the invention may be used to output basic metrics, a barcode rank plot, a clustering map, a differential expression table, or combinations thereof. A directory may be created for each or any set of called cells e.g., within a directory such as <output-root>/clustering, containing the following:
A csv file listing the cluster assignment of each cell from graph-based clustering and from all the K-means runs.
A csv file containing the silhouette scores for graph-based clustering and for all the K-means runs. The silhouette score is a measure of clustering quality that takes into account both intra-cluster and inter-cluster distances. Note that it is calculated in the PCA space used for clustering and not in the visualized UMAP space.
UMAP plots for graph-based clustering and for all the K-means runs. UMAP (Uniform Manifold Approximation and Projection) is a popular method for projecting multi-dimensional data onto a 2-D space.
csv files for graph-based clustering and for all the K-means runs containing the top-ranked genes for each cluster.
csv files similar to the top genes files, but which include additional statistics for each gene: Z-score, p-value, adjusted p-value and log2 fold change.
A csv file with cell type assignments for each clustering type (when applicable).
A *.png file with cell type maps for each clustering type (when applicable).

The plots below show clustering results for HEK/3T3 and PBMC samples. The number of clusters shown in each case is the one which produced the highest silhouette score.

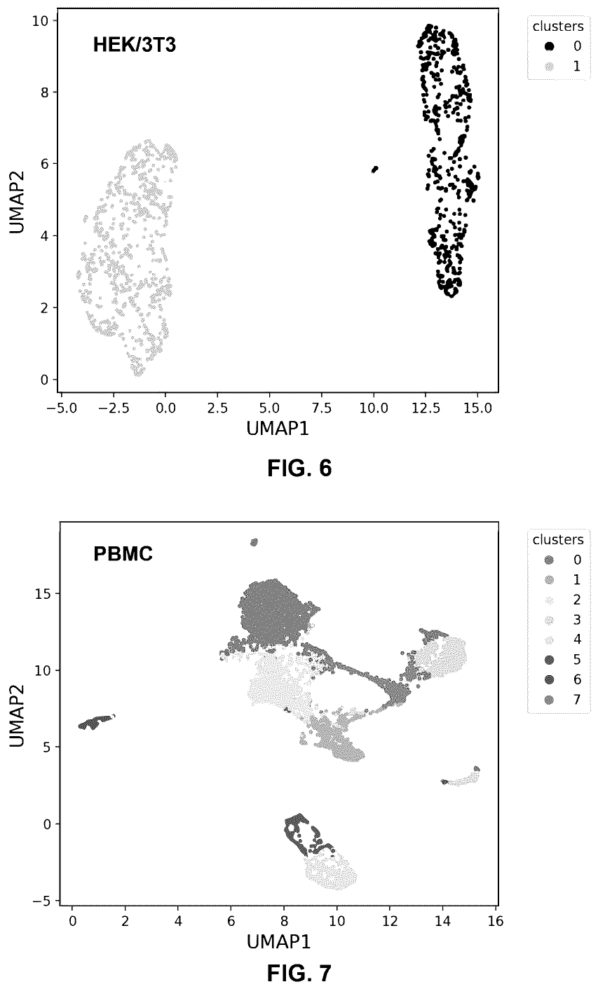

FIG. 6 shows a clustering result for an HEK/3T3 sample.

FIG. 7 shows a clustering result for a PBMC sample.

### 8. Metrics Extraction

In the final step of the analysis, various metrics are calculated and reported for each sensitivity level (or for a single set when the number of cells is specified by the user).

In some embodiments, systems of the invention may include one or any combination of a number of basic metrics. For example, one or more of the following metrics may be included in every analysis:
Total number of reads in the input fastq files
Percentage of mapped reads (Note that the denominator does not include excluded reads from low count barcodes and reads that were too short after trimming).
Number of called cells
Mean reads per cell (This is the total number of input reads divided by the number of cells).
Percentage of reads in cells (This is the number of reads associated with cell-containing barcodes divided by the total number of input reads).
UMI duplication rate in cells (This is the number of mapped reads in cells divided by the number of unique reads (duplicated reads from the same transcript) in cells For example, if every UMI had three reads containing it, i.e., two deduplicated reads per UMI, the UMI duplication rate would be 3).
Total number of UMIs in cells
Median UMIs per cell (This is based on the distribution of counts of unique UMIs (transcripts) associated with each cell barcode).
Number of genes expressed in cells (This number includes all genes with a nonzero count in the filtered matrix, and gives equal weight to every gene regardless of expression level).
Median genes per cell (This number includes all genes with a nonzero count in the filtered matrix, and gives equal weight to every gene regardless of expression level).
Percentage of cells with more than 50% of transcripts being from mitochondrial genes
Percentage of cells with less than 5% of transcripts being from mitochondrial genes

### Barnyard Metrics and Estimation of Multiplet Rate

Embodiments of the system provide tools useful to assess the quality of an scRNA-Seq protocol such as Drop-seq or inDrop or a particular chemistry or reaction setup in on of those protocols. One way to assess the quality of a single cell RNA technology is a combined species (“barnyard”) experiment, typically involving a mixture of human and mouse cells. Such an experiment can provide information about the prevalence of multiplets, or beads that capture more than a single cell. It can also assess the degree of interference from background RNA by measuring cross-species contamination. The more human genes found in mouse cells and vice versa, the more background contamination is likely present in the system.

When the reference transcriptome is a combined human and mouse transcriptome, systems of the invention are operable to provide an additional set of metrics specific to a barnyard experiment:
Number of human cells (This is the number of cell barcodes with at least 85% of the transcripts coming from the human reference).
Number of mouse cells (This is the number of cell barcodes with at least 85% of the transcripts coming from the mouse reference).
Number of multiplets (This is the number of cell barcodes with less than 85% of the transcript coming from the human or the mouse reference; Note that multiplets can consist of two human cells or two mouse cells, or (rarely) include more than two cells, so this probably underestimates the true number of multiplets).
Total number of UMIs in human cells
Median human UMIs per human cell
Number of human genes expressed in human cells
Median human genes per human cell
Percentage of mouse UMIs in human cells
Total number of UMIs in mouse cells
Median mouse UMIs per mouse cell
Number of mouse genes expressed in mouse cells
Median mouse genes per mouse cell
Percentage of human UMIs in mouse cells

An important output of a barnyard analysis is called a barnyard plot.

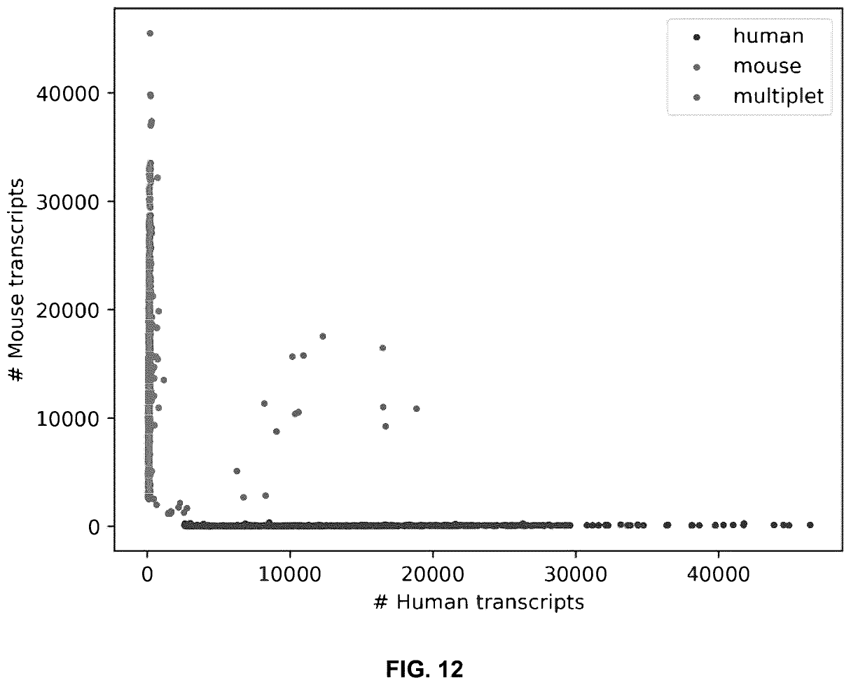

FIG. 12 is a barnyard plot that shows the number of mouse transcripts vs. the number of human transcripts, with different darkness indicating human cells, mouse cells and multiplets:

### 9. Outputs and Report Generation

After all the metrics are calculated, results for each set of called cells may be reported in files such as the following:
Basic metrics are stored in csv and json formats in <output-root>/metrics.
If applicable, barnyard metrics are stored in csv and json format in <output-root>/barnyard.

### Analysis Reports

Operating systems of the invention to analyze sequence data from an scRNA-Seq experiment optionally produced a report, such as a document in a format such as XML or HTML. Such reports may be stored in a suitable directory, such as:
<output-root>/report, and may include basic metrics, a barcode rank plot, a clustering map and a differential expression table. The user can toggle between different clustering modes—
graph-based and K-means at different K values, which will change the content of the map and the table If “barnyard” mode is used, those statistics and a barnyard plot will be included as well.

In a standard analysis, a report may be generated for each sensitivity level. An additional combined report (e.g., “combined.html”) may include all sensitivity levels. The user may be given the option to browse through the different sensitivities in the combined report and select the optimal sensitivity level or decide on a number of cells to force in a subsequent reanalysis.

In the case of an analysis with a manually selected (forced) number of cells, a report will be generated for this specific number, and a new tab may be added to the combined report.

### Running the Pipeline

Preferred embodiments of the system operate in a computer system that includes at least one processor coupled to a memory subsystem. The functionality provided by the system may be implemented in a local computer, a server system, or a cloud-based computing system. Software modules to perform the described functionality may be developed in any suitable environment such as, for example, python, Ruby on rails, C++, others, or combinations thereof. Versions of systems of the invention are executable in mac osx, Linux, and windows operating systems. Preferred embodiments operate in server or cloud environments and are operable to receive sequence data, e.g., in a FASTQ or FASTA format, from a sequencing resource such as a next-generation sequencing (NGS) instrument or a genomics facility. The system may align reads to a reference generating a sequence alignment map (SAM) or binary alignment map (BAM) and provide outputs to a user computer. Details and variations of embodiments of functionality of the system are described herein.

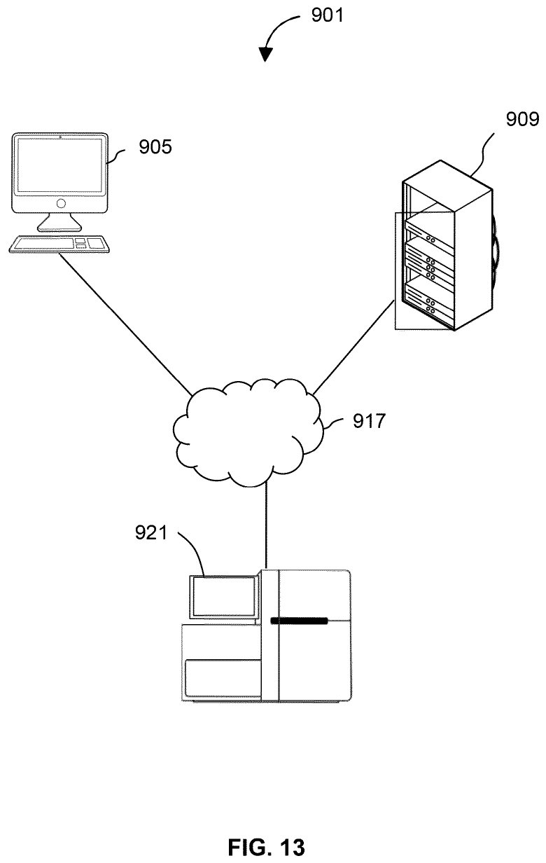

FIG. 13 shows a system 901 that includes at least one local computer 905. The system 901 may also include a server or cloud computer 909. Preferably any local computer 905 or server or cloud computer 909 included in the system 901 includes at least one processor coupled to a memory subsystem. Preferably any local computer 905 or server or cloud computer 909 included in the system 901 is operable to receive sequence data from a sequencing instrument 921 or genomics facility over communication network 917, by which machines of the system 901 communicate and interoperate with one another. The memory subsystem in the local computer 905 or server or cloud computer 909 of the system 901 preferably includes program instructions executable by the processor to cause the system 901 to perform analytical functions described herein.

Thus, systems of the invention provide complete analysis solutions for a single sample. Systems of the invention may take in a paired-end set of fastq files, and returns a set of outputs including a full barcode x gene count matrix, a set of filtered count matrices for different cell calling sensitivity levels and various sample metrics. For mixed human/mouse samples (“barnyard” experiments), the results can include specific metrics related to species separation. The combined results are summarized in HTML reports. In certain embodiments, systems of the invention run from the command line prompt on Linux, Windows and Mac. Note that file and directory names containing spaces may be enclosed in quotation marks.

See below a description of all the required and optional arguments for certain embodiments of systems of the disclosure. To see the full list of arguments from the command line, run:

$ pipseeker count --help.
Required arguments
$ pipseeker count --input-path <path to fastq files> --output-
root <destination for all outputs> --STAR-index-path <path to
mapping reference>
--input-path (string)

The path to the source fastq files. If this is a directory name, all files with names ending with .fastq or fastq.gz will be included as inputs. Alternatively, if there are files from multiple samples in one directory, you can specify a prefix at the end of the path, which will restrict the files included only to ones whose name begins with that prefix.

For example, consider a situation where the input directory contains two-lane results from two different samples:

| $ ls my_output_dir | |
|---|---|
| sample_1_L001_R1.fastq.gz | sample_1_L001_R2.fastq.gz |
| sample_1_L002_R1.fastq.gz | sample_1_L002_R2.fastq.gz |
| sample_2_L001_R1.fastq.gz | sample_2_L001_R2.fastq.gz |
| sample_2_L002_R1.fastq.gz | sample_2_L002_R2.fastq.gz |

Using --input-path my_output_dir will result in the undesirable situation of all files being included in the analysis as components of the same sample. Instead, use --input-path my_output_dir/sample_1 and --input-path my_output_dir/sample_2 in two separate runs to include only the relevant files for each sample.

--output-root (string)

The directory where all PIPseeker outputs will be stored. It can be the same as the input path or a different directory.

--star-index-path (string)

The path to a directory containing an indexed reference to use for mapping.

### Optional Arguments

General:
--chemistry (string, default: mark6)

Allows user to input chemistry used to produce the sample. Accepted values are specific to an embodiment (e.g., mark3 and mark6).

--downsample-to (integer) )

An integer representing the target number of reads to be mapped if downsampling is desired. If specified, reads will be selected randomly and only the selected subset will be fed into STAR for mapping.

--input-reads (integer)

An integer representing the number of input reads if --downsample-to is specified. In order to calculate the downsampling ratio, the total number of reads has to be known in advance.

--input-reads should include the total number of reads combined across all input files. If it is not specified, reads will be counted manually, so using this argument can reduce running time.

```
--verbosity (integer, default: 1)
    Verbosity level: 0 (silent), 1 (concise), or 2 (detailed).
Barcoding
  --min-reads-per-barcode (integer, default: 1)
```

The minimum number of reads associated with a barcode. If a barcode is associated with fewer reads than the specified number, all reads from this barcode will be discarded from further analysis. Increase this number to reduce mapping time by not processing barcodes unlikely to be considered cells. Note that the low-count portion of the barcode rank plot will be incomplete and mapping statistics may change slightly.

--retain-barcoded-fastqs

A flag for retaining the intermediate, barcoded fastqs. In most cases it is not needed. If not specified, those fastqs will be discarded after completion of the analysis. Note that the input fastqs are always retained, regardless of this flag.

Mapping
--star-threads (integer, default: 8)

Number of CPU threads to use for STAR mapping (default: 8).

Cell calling
--force-cells (integer)

Force a specific number of barcodes to be considered as cells. If specified, this number of barcodes will be selected from the top of the barcode rank plot. This will replace the default, sensitivity-based mode of cell calling.

Clustering
--clustering-percent-genes (integer, default: 10)

Percentage of genes to use for clustering. Genes with the highest variability in expression up to this percentile rank will be included.

--principal-components (integer, default: 15)

The number of components for PCA dimensionality reduction.

--percent-genes (float, default: 10)

The percentage of genes to select for clustering based on highest variance in expression.

--min-clusters-kmeans (integer, default: 2)

Minimum number of clusters for K-means clustering. The values of K will be incremented starting at this number.

--max-clusters-kmeans (integer, default: 12)

Maximum number of clusters for K-means clustering. The values of K will be incremented until reaching this number.

--nearest-neighbors (integer, default: 10)

Number of nearest neighbors for graph-based clustering. This defines the minimum number of cells per cluster.

--diff-exp-genes (integer, default: 50)

Number of top differentially expressed genes to include for each cluster.

Metrics
--run-barnyard

A flag to enable “barnyard” metrics to be generated and included in the analysis report.

Report
--id

Sample ID to include in the output report.

--description

Sample description to include in the output report.

### Re-Running the Final Stages: Reanalyze

After observing the results of a complete analysis, a user may wish to change a parameter related to the late stages, such as the range of K values for K-means clustering. The user may also wish to force a certain number of barcodes to be assumed to represent cells. This should not require repeating the barcoding and mapping steps, which are typically the most time consuming.

Using the reanalyze function will, in preferred embodiments, skip barcoding and mapping and start the analysis directly at cell calling. If this mode is used, input-path should point to a directory containing a full analysis result from a previous run. --output-root, --chemistry. Arguments related to barcoding and mapping will be ignored.

For example, assume that after running the full pipeline and visually inspecting the results, none of the standard cell calling sensitivity levels seem to capture the correct region of the barcode rank plot. A user may force a specific number of cells without re-running the entire pipeline:

$ pipseeker reanalyze --input-path my_analysis_result_dir --
force-cells 5000

Using the described commands, hardware, and functionality, the invention provides a system comprising a processor coupled to a memory subsystem comprising instructions executable by the processor to cause the system to evaluate gene expression levels from scRNA-Seq experiments. The system uses in silico cell calling tools for distinguishing between partitions that contain cells and background partitions that did not fully capture a cell. The system also provides barcode processing tools that tests barcodes from at least cellular and UMI tiers against a whitelist and optionally converts those to a compact index format. Further, the system can implement multimapping tools to preserve information for reads that map to multiple locations in a reference. The system also contains analytics tools for clustering, cell classification, and providing reports that include, e.g., measures of expression levels. The functionality of the system addresses issues brough to light by scRNA-Seq using pre-templated instant partitions (PIPs) in which very large numbers of cells are isolated into droplets simultaneously with a rapidity not reached by legacy microfluidics.

What is claimed is:

1. A method of identifying cell types, the method comprising:
obtaining gene expression data for a plurality of cells;
assigning the cells to clusters based on the gene expression data;
determining non-parametric statistical scores of differential gene expression for each cluster versus other clusters;
calculating a correlation between the calculated score and annotated clusters in a reference; and
annotating each cluster with a cell type based on correlations to the annotated clusters.

2. The method of claim 1, wherein the determining step comprises performing a Mann-Whitney test to calculate a U score for genes of each cluster.

3. The method of claim 2, wherein the annotated clusters in the reference comprise reference U scores

4. The method of claim 1, wherein the plurality of cells include peripheral blood monocytes and the clusters are annotated with cell types that include one or more of B cells, CD4 memory T cells, CD4 naïve T cells.

5. The method of claim 1, wherein the assigning step is performed by obtaining a principal component analysis (PCA) matrix of the gene expression data; creating a k-nearest neighbor graph from the PCA matrix; and performing Leiden clustering on the graph.

6. The method of claim 1, wherein the assigning, determining, calculating, and annotating are performed by a computer system comprising at least one processor coupled to a memory subsystem containing instructions executable by the processor to cause the computer system to perform the recited operations.

7. The method of claim 6, wherein the reference includes the annotated clusters of reference cells of known type, wherein the annotated clusters were created by identifying reference genes with the highest differential expression among the reference cells and clustering the reference cells into the annotated clusters based on those reference genes.

8. The method of claim 7, wherein the reference further includes reference U scores calculated by a non-parametrical statistical test for each annotated cluster.

9. The method of claim 8, wherein the reference U scores are calculated by the computer system as the Wilcoxon ranked sum.

10. The method of claim 1, wherein each cluster is given an annotation in one of three categories that include (i) definitive, (ii) primary and secondary, and (iii) no assignment.

11. The method of claim 6, further comprising performing the obtaining step by single-cell RNA-sequencing (scRNA-seq) to obtain scRNA-seq data and performing the determining step to produce the non-parametric statistical scores of differential gene expression for each cluster from the scRNA-seq data.

12. The method of claim 1, wherein the obtaining step comprises performing scRNA-Seq in droplets.

13. The method of claim 12, further comprising
combining template particles with cells in a first fluid wherein the template particles are each linked to copies of a capture probe;
shearing the first fluid in the presence of a second immiscible fluid to thereby generate a plurality of droplets substantially simultaneously, wherein at least one droplet contains a single template particle and a single target cell;
lysing the target cell to release mRNA molecules in the droplet; and
capturing the mRNA molecule in the droplet with the capture probes of the single template particle.

14. The method of claim 13, further comprising performing reverse transcription to yield a cDNA using a reverse transcriptase (RT).

15. The method of claim 14, wherein the RT adds additional bases to a 3′ end the cDNA.

16. The method of claim 14, wherein the reverse transcription leaves the cDNA linked to bead, the cDNA comprising first and second universal primer binding sites, at least one cell barcode, and at least one unique molecular identifier (UMI).

17. The method of claim 13, wherein the capture probe comprises one or more of a template switching segment, a UMI, a droplet barcode, and a universal primer nucleotide sequence.

18. The method of claim 13, wherein the first fluid is an aqueous fluid and the second fluid comprises an oil.

19. The method of claim 18, wherein the template particles further comprise one or more compartments, recesses, or pores that contain one or more of a cell lysis reagent, a nucleic acid synthesis reagent, and a capture tag or oligonucleotide.
